(19) United States
(12) Patent Application Publication
(10) Pub. No.: US 2025/0161367 A1
(43) Pub. Date: May 22, 2025

(54) THERAPEUTIC USE OF FIBROBLASTS TO STIMULATE REGENERATION AND REPAIR OF ATROPHIED SPLEEN

(71) Applicant: FIBROBIOLOGICS, INC., Houston, TX (US)

(72) Inventors: Pete O'HEERON, Houston, TX (US); Hamid KHOJA, Houston, TX (US)

(73) Assignee: FIBROBIOLOGICS, INC., Houston, TX (US)

(21) Appl. No.: 18/841,921

(22) PCT Filed: Feb. 2, 2023
(86) PCT No.: PCT/US2023/061822

§ 371 Date: Aug. 27, 2024

Related U.S. Application Data
(60) Provisional application No. 63/383,500, filed on Nov. 13, 2022.
(60) Provisional application No. 63/344,691, filed on May 23, 2022.
(60) Provisional application No. 63/316,264, filed on Mar. 3, 2022.

(51) Int. Cl. A61K 35/33; A61K 35/17; A61K 39/02; A61K 39/12; A61K 39/39; C12N 5/077

(52) U.S. Cl. CPC A61K 35/33; A61K 35/17; A61K 39/02; A61K 39/12; A61K 39/39; C12N 5/0656

(57) ABSTRACT

Embodiments of the present disclosure concern administration of a cell population to an individual for regeneration of a tissue. The cell population may comprise fibroblasts and/or immune cells, either of which may be modified or unmodified. The a tissue for regeneration may be a spleen.

This application claims priority to U.S. Provisional Patent Application Ser. No. 63/316,264, filed Mar. 3, 2022, and also claims priority to U.S. Provisional Patent Application Ser. No. 63/344,691, filed May 23, 2022, and also claims priority to U.S. Provisional Patent Application Ser. No. 63/383,500, filed Nov. 13, 2022, all of which applications are incorporated by reference herein in their entirety.

### BACKGROUND

### I. Technical Field

This disclosure relates at least to the fields of medicine, immunology, cell biology, and molecular biology.

### II. Background

Fibroblasts are no longer considered as mere structural components of organs but as dynamic participants in immune processes. Fibroblasts produce an environment that influences T regulatory cell migration, proliferation, and activity to ensure immunotolerance [1].

The spleen is one of the key secondary lymphoid organs responsible for the rapid response of the immune system to pathogens in the blood, and to maintain a long term adaptive response to such pathogens. The spleen also serves as the key organ for iron metabolism and erythrocyte homeostasis. The organ also functions as key storage site for platelets and leukocytes. A verity of cells have been identified in the spleen, including, endothelia cells, mesothelial cells, reticular cells, erythrocytes, granulocytes, mononuclear cells, hemopoietic cells, macrophages, dendritic cells, plasma cells, CD4+ and CD8+ T cells, and migrating B cells. With age, the structure and function of the spleen changes, leading to decreased ability to respond positively to vaccination, increased susceptibility to viral and bacterial pathogen infections, and increased incidence of autoimmune disease [2].

With an increase in lifespan, and a greater percentage of aged in the population, there is a need for improving and extending the productive life of the spleen through cell therapy using fibroblasts and their interactions with the other cells in the spleen.

### BRIEF SUMMARY

Embodiments disclosed herein concern compositions and methods for regenerating a tissue in an individual. In some embodiments, one or more compositions is administered to an individual, including for the purpose of regenerating the tissue, stimulate expansion of necessary cells for critical organ function, and/or reinvigorating production of cellular products necessary for reversing organ atrophy, including splenic atrophy. The administration may be useful for regenerating a tissue, such as a spleen tissue. In some embodiments, the regenerating comprises organogenesis and/or T cell development and/or B cell development. In some embodiments, wherein the tissue comprises the spleen, the regenerating comprises reversing of splenic atrophy. In some embodiments, the regenerating comprises tissue differentiation, such as splenic differentiation. In some embodiments, the regenerating comprises tissue recruitment, differentiation of stems cells into epithelia cells of the tissue and/or fibroblasts of the tissue, and/or expansion of epithelia cells of the tissue and/or fibroblasts of the tissue. In certain embodiments, all or a combination of at least some of the aforementioned activities are encompassed in regenerating a tissue. The fibroblasts of the tissue may comprise splenic reticular fibroblasts, in some cases.

Certain embodiments concern cell populations and their use as cellular therapies. The cell population may comprise modified and/or unmodified fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells). The cell population may comprise modified and/or unmodified immune cells. The cell population may comprise fibroblast derivatives (including extracellular vesicles including exosomes, microvesicles, apoptotic bodies or any other fragments or biologic components of fibroblast cells). In some embodiments, the cell population comprises unmodified fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells), unmodified immune cells, activated fibroblasts, activated immune cells, fibroblast derivatives, immune cell derivatives, or a combination thereof. The fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells) and/or immune cells may be activated by exposing the cells to one or more agents capable of activating the cells. The fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells) and/or immune cells may be activated by exposing the cells to one or more agents capable of activating the cells, thereby producing activated fibroblasts and/or activated immune cells, respectively. In some embodiments, the agent(s) comprise one or more nucleic acids, cytokines, chemokines, transcription factors, epigenetic factors, growth factors, hormones, or a combination thereof. The population of cells may be activated in vitro or ex vivo.

In some embodiments, fibroblasts are activated in vivo, ex vivo, or a combination of both, by selecting fibroblast cells which express and release of, or activated to express one of more of factors such as CCL21, podoplanin, CCL19, IL-7, IL-33, DES, MAdCAM-1, CXCL13, BAFF, IL-6, [3-10]

In some embodiment, activation methods with RNA, and or transcription factor modulation, Fibroblasts can be activated to express transcription factors such as SPIB, WT1, STAT1, STAT2, IRF9(ISFG3), ETS[11, 12][13-16]

In some embodiment, activation methods involve the activation of fibroblasts with RNA and or transcription factors to ensure an increase in the expression and secretion of Bst2, CXCL9, CXLX10, Ly6c, Sca-1, Timd4, Fabp5 [13, 17]

In some embodiments, RNA and or epigenetic modulation could be used to, in vivo, or ex vivo, increase the expression of one or a combination of two or more of fibroblast associated genes such as Col1a1, Col3a1, Col6a1, Dnc, Lum, Mgp, Sparc, Speringl, Serpinh1, Htra1, Htra3, Mmp2, and or Mmp3[18].

In some embodiments, the cell population is autologous, allogeneic, xenogeneic, syngeneic, or the cell population may comprise a combination thereof, with respect to the individual.

In some embodiments, the cell population is administered to the individual systemically and/or locally to the spleen of the individual, including at least an individual in need of regeneration of at least part of the spleen.

Certain embodiments concern methods, including in vitro methods, for producing activated cell populations, such as activated fibroblasts, derivatives of activated fibroblasts (including extracellular vesicles including exosomes, microvesicles, apoptotic bodies or any other fragments or biologic components of fibroblast cells), activated immune cells, derivatives of activated immune cells, or a combination thereof. In some embodiments, the method comprises the step of exposing fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells) and/or immune cells to one or more conditions that induce activation. The activation of the fibroblasts, derivatives of activated fibroblasts (including extracellular vesicles including exosomes, microvesicles, apoptotic bodies or any other fragments or biologic components of fibroblast cells), activated immune cells, and/or derivatives of activated immune cells may make the cell population able to regenerate a spleen. The activated cells may be autologous, allogeneic, xenogeneic, syngenetic, or the population may comprise a combination thereof, with respect to the individual. In some embodiments, the cells are activated by conditions comprising one or more nucleic acids, cytokines, chemokines, transcription factors, epigenetic factors, growth factors, hormones, or a combination thereof.

In some embodiments, the regeneration of the tissue comprises stimulating, improving, or increasing production and/or activity of immune cells in a spleen.

Embodiments of the disclosure include methods of regenerating a tissue in an individual, comprising the step of administering an effective amount of a population of cells to an individual; wherein the tissue comprises the spleen of the individual; wherein the population of cells comprises unmodified or modified fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells), unmodified or modified immune cells, activated or non-activated fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells), activated or non-activated immune cells, fibroblast derivatives (including microvesicles, apoptotic bodies or any other fragments or biologic components of fibroblast cells), immune cell derivatives, or a combination thereof, thereby resulting in regeneration of spleen tissue in the individual. In some cases, prior to and/or during the administering, the population of cells is activated by exposing the cells to one or more agents capable of activating the cells. The agent(s) may comprise one or more nucleic acids, cytokines, chemokines, transcription factors, epigenetic factors, growth factors, hormones, or a combination thereof. In some cases, the population of cells is activated in vitro, in vivo, or ex vivo, and the population of cells may be autologous, allogeneic, xenogeneic, syngeneic, or a combination thereof, with respect to the individual. For any method, one step may further comprising administering one or more adjuvants to the individual, such as a chemical adjuvant, a viral adjuvant, a bacteria adjuvant, or a combination thereof. In any case, the adjuvant may comprise an encapsulated RNA, a microRNA, an siRNA, or a combination thereof.

In certain embodiments, regenerating comprises organogenesis and/or T cell development and/or B cell development. In certain embodiments, regenerating comprises reversing of splenic atrophy. In certain embodiments, regenerating comprises reinvigorating or stimulating of spleen immune cell production. In certain embodiments, regenerating comprises tissue differentiation. In certain embodiments, regenerating comprises tissue recruitment, differentiation of stems cells into epithelia cells of the tissue and/or fibroblasts of the tissue, and/or expansion of epithelia cells of the tissue and/or fibroblasts of the tissue.

In specific embodiments of any composition and/or method, fibroblasts of the tissue comprise splenic reticular fibroblasts.

In methods in which an activated population of cells is administered to one or more individuals, it may be administered systemically to the individual or locally to the spleen of the individual.

In particular embodiments, there is an in vitro method of producing activated or non-activated fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells), derivatives of activated fibroblasts (including extracellular vesicles including exosomes, microvesicles, apoptotic bodies or any other fragments or biologic components of fibroblast cells), activated or non-activated immune cells, derivatives of activated or non-activated immune cells, or a combination thereof, comprising the step of exposing fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells) and/or immune cells to conditions that induce activation, wherein the activated fibroblasts, derivatives of activated fibroblasts (including extracellular vesicles including exosomes, microvesicles, apoptotic bodies or any other fragments or biologic components of fibroblast cells), activated immune cells, and/or derivatives of activated immune cells comprise activity for regenerating spleen tissue. The activated or non-activated fibroblast cells (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells) and/or activated or non-activated immune cells may be autologous, allogeneic, xenogeneic, or syngeneic in relation to the spleen. In certain embodiments, regeneration comprises stimulating, improving, and/or increasing production and/or activity of immune cells in a spleen. For the methods, the conditions may comprise one or more nucleic acids, cytokines, chemokines, transcription factors, epigenetic factors, growth factors, hormones, or a combination thereof.

In one embodiment, there is a method of stimulating, improving, or increasing production and/or activity of cells in a spleen in an individual, comprising the step of providing to the individual an effective amount of activated fibroblasts, derivatives of activated fibroblasts, activated immune cells, derivatives of activated immune cells, or a combination thereof.

It is contemplated that any embodiment discussed in this specification can be implemented with respect to any method or composition of the disclosure, and vice versa. Furthermore, compositions of the disclosure can be used to achieve methods of the disclosure.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term “about” means within an acceptable error range for the particular value.

As used herein, the term “activated immune cells” refers to immune cells treated with one or more stimuli capable of inducing one or more alterations in the cell: metabolic, immunological, epigenetic, growth factor secreting, surface marker expression, and production and excretion of microvesicles.

The term “administered” or “administering”, as used herein, refers to any method of providing a composition to an individual such that the composition has its intended effect on the patient. For example, one method of administering is by an indirect mechanism using a medical device such as, but not limited to a catheter, applicator gun, syringe etc. A second exemplary method of administering is by a direct mechanism such as, local tissue administration, oral ingestion, transdermal patch, topical, inhalation, suppository etc.

As used herein, “allogeneic” refers to tissues or cells from another body that in a natural setting are immunologically incompatible or capable of being immunologically incompatible, although from one or more individuals of the same species.

As used herein, “autologous” refers to tissues or cells that are derived or transferred from the same individual's body (i.e., autologous blood donation; an autologous bone marrow transplant).

As used herein, “agent” refers to nucleic acids, cytokines, chemokines, transcription factors, epigenetics factors, growth factors, or hormones.

As used herein, “xenogeneic” refers to tissues or cells from a species different from the patient.

“Cell culture” is an artificial in vitro system containing viable cells, whether quiescent, senescent or (actively) dividing. In a cell culture, cells are grown and maintained at an appropriate temperature, typically a temperature of 37° C. and under an atmosphere typically containing oxygen and CO₂. Culture conditions may vary widely for each cell type though, and variation of conditions for a particular cell type can result in different phenotypes being expressed. The most commonly varied factor in culture systems is the growth medium. Growth media can vary in concentration of nutrients, growth factors, and the presence of other components. The growth factors used to supplement media are often derived from animal blood, such as calf serum.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The term “individual”, as used herein, refers to a human or animal that may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” may be used interchangeably and refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “unmodified” as used herein when referring to cells or a cell population may refer to cells that have only been cultured under standard conditions for the cell type, without exposure to any agent capable of changing the intrinsic characteristics (except those that may inherently change during a standard cell culture) of the cell type. An unmodified cell comprises only endogenous genetic material, i.e. an unmodified cell comprises no transgenes. In some embodiments, it refers to cells that have not been manipulated in any way or that have only been cultured. In a specific embodiment, “modified” cells are cells that have been stimulated to a point that they have differentiated into another cell type and may no longer have the morphology of the starting material. This can be applicable to MSCs and fibroblasts, as examples.

As used herein, the term “transplantation” refers to the process of taking living tissue or cells and implanting it in another part of the body or into another body.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

### II. Fibroblasts

Embodiments of the disclosure encompass compositions, methods, and/or systems for introducing fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells) to the spleen and/or modifying the existing fibroblast cells (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells) within the spleen for regeneration of its function [3, 4]. In particular embodiments, the disclosure concerns interaction of a first type of cell with a second type of cell and/or certain agent(s). The interaction may include one or more modifications to the first and/or second type of cell as a result of the interaction. Embodiments of the disclosure include compositions, methods, and systems in which fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells) are modified upon exposure to certain cells and/or one or a combination of certain agents including nucleic acids, cytokines, chemokines, or growth factors. In some embodiments, introduction of these modified or unmodified fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells) into the spleen regenerates necessary functional cells, such as splenic reticular fibroblast cells, splenic fibroblasts, splenic macrophages, stromal organizer cells, and/or other non-hemopoietic cells. In some embodiments, the introduction initiates the foundation for new tissue growth [5]. The regeneration of these cells can be either through several possible steps such as: the differentiation of the modified or unmodified administered fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells) into reticular fibroblasts, splenic fibroblasts, stromal organizer cells, and/or discontinuous splenic epithelium; activation of the senescent splenic reticular epithelium, fibroblasts, and/or stromal organizer cells, including through cell to cell contact with the modified or unmodified fibroblasts; activation of senescent splenic reticular epithelium, fibroblasts, and/or stromal organizer cells by products excreted from the modified or unmodified fibroblasts in the splenic microenvironment; differentiation of local stem cells into splenic reticular fibroblast cells, splenic fibroblasts, splenic macrophages, and/or stromal organizer cells including through the direct/indirect action of the injected modified/unmodified fibroblasts and/or via excreted products from the modified/unmodified fibroblasts; and/or expansion of the small number of splenic reticular fibroblast cells, splenic fibroblasts, splenic macrophages, stromal organizer cells remaining in the injured or age-related atrophy impacted spleen to sufficient numbers, which may impact and significantly improve the function of the spleen.

In some embodiments, reversal of the splenic atrophy is monitored by magnetic resonance imaging (MRI), such as to determine the fat-free fraction of spleen. In addition, complete blood count assessment, fluorescence-activated cell sorting (FACS) sorting characterization of the blood cells, and/or flow cytometry may provide metrics necessary to determine changes in the spleen and/or monitor the impact of injected modified and/or unmodified fibroblasts into the atrophied spleen.

Certain embodiments concern compositions, methods, and/or systems in which certain cells, including any spleen cell disclosed herein, are modified upon exposure to fibroblasts and/or certain agents excreted from fibroblasts. In particular embodiments, the interaction of fibroblasts with one or more other types of cells (and optionally that interaction also includes one or more certain agents) results in modification of the fibroblasts and/or one or more cells of the spleen [6]. In specific embodiments, cells of the spleen include at least immune cells.

In certain embodiments, introduction of fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells) to the spleen results in the modification of fibroblasts and/or other cells found in the spleen to stimulate regeneration of the splenic structure and function [7]. In certain aspects, exposure of fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells) and/or agents produced therefrom result in modifications to one or more types of immune cells and/or exposure of one or more types of immune cells and/or agents produced therefrom result in modification to the fibroblasts. In specific embodiments, one or more agents are also included in the exposure and may or may not be exogenously provided, such as in other cases where they are endogenous to an environment and/or cell and/or tissue.

In specific embodiments, methods of the disclosure occur in vitro or ex vivo, such as in a culture. In particular cases, the methods occur by the hand of man and do not encompass ordinary or random occurrences in a body. The methods of the disclosure are non-natural, in particular aspects. In specific embodiments, the concentrations of cells used in a method of exposing one type of cells to another type of cells does not occur in nature and does not happen randomly in nature. In specific embodiments, the concentration of one or more agents used in a method of exposing the one or more agents to one or more types of cells does not occur in nature and does not happen randomly in nature. The modification of any types of cells encompassed by the disclosure that occurs ex vivo or in vitro does not occur in vivo naturally in the same manner. In specific embodiments, tissue biopsy from the donor is use to isolate, characterize, if necessary activate, expand, and reintroduce back into the donor one or a combination of the cells for the purpose of reactivating atrophied spleen [8].

The disclosure encompasses therapeutic uses of cells, including epithelial cells, thymocytes, dendritic cells, macrophages, B lymphocytes, myoid cells, endothelial cells, fibroblasts, immune cells, splenic reticular fibroblast cells, splenic fibroblasts, splenic macrophages, stromal organizer cells, and mixtures thereof. In at least some cases, the fibroblasts have been modified prior to their exposure to one or more immune cells and other cells found in the spleen, such as chemically, physically, and/or epigenetically activated, or exposed to conditions that are not normally found in the body. In certain embodiments, immune cells, or their derivatives, have been modified, such as activated, prior to their exposure to the fibroblasts.

Embodiments of the disclosure provide means of utilizing fibroblasts (including fibroblast cells, fibroblast-like cells, and/or extracellular vesicles including exosomes, microvesicles, apoptotic bodies or any other fragments or biologic components of fibroblast cells) as allogeneic, autologous, xenogeneic, or syngeneic therapeutic cells through modification of culture conditions. In one embodiment of the disclosure, fibroblasts are extracted from sources with lower immunogenicity (e.g. placental fibroblasts, omental tissue derived fibroblasts, cord blood derived fibroblasts, etc.).

Embodiments of the disclosure provide methods of reducing immunogenicity of particular types of fibroblasts. Fibroblasts may be derived from various tissues or organs, including, but not limited to skin, heart, blood vessels, bone marrow, skeletal muscle, liver, pancreas, brain, foreskin, which can be obtained by biopsy (where appropriate) or upon autopsy. In some aspects, the cells comprise fibroblasts, which can be from a fetal, neonatal, adult origin, or a combination thereof.

Fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells) for use in any methods of the disclosure may be exposed to certain medium component(s), in specific embodiments.

Certain embodiments concern methods of producing activated or unactivated fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells) or activated or unactivated immune cells or activated or unactivated immune cell or fibroblast derivatives for introduction into the spleen. Introduction in the spleen may stimulate, improve, and/or increase production or activity of immune cells, iron metabolism, and/or erythrocyte homeostasis. In some embodiments, the method is performed in vivo. In some embodiments, the method is performed in vitro. In some embodiments, the method is performed ex vivo.

In some embodiments, the fibroblast cells are autologous. In some embodiments, the fibroblast cells are allogeneic. In some embodiments, the fibroblast cells are xenogeneic.

Certain embodiments concern introduction of fibroblasts (including fibroblast cells, fibroblast-like cells, lysate from fibroblasts, conditioned media from fibroblast culture, and/or extracellular vesicles including exosomes, microvesicles, apoptotic bodies or any other fragments or biologic components of fibroblast cells), fibroblasts activated with an agent, and/or fibroblasts plus one or more type of immune cells and/or derivative agents into a spleen. The spleen may be atrophied. The introduction may induce organogenesis and T cell development, and/or reversing of splenic atrophy. The introduction may induce tissue differentiation for the reversing of splenic atrophy [14]. The introduction may induce cell recruitment and differentiation of stems cells into the spleen red pulp, white pulp, or marginal zones [12]. The introduction may induce expansion of already locally present splenic endothelial cells, mesothelial cells, reticular cells, reticular fibroblast cells, splenic fibroblasts, splenic macrophages, stromal organizer cells [15-17].

Certain embodiments concern the use of one or more adjuvants in combination with fibroblasts to activate the immune system. The adjuvants may be chemical, viral, or bacterial product based.

Certain embodiments concern a combination of encapsulated RNA, microRNA, or other RNAi molecules or systems with fibroblasts. The combination may locally activate splenic tissue regeneration or reverse splenic atrophy.

### III. Agents

Certain embodiments concern certain agents and methods for using such agents. The agents may comprise any agent useful for the methods and compositions encompassed herein. Cell populations of the present disclosure may be exposed to one or more agents for one or a variety of purposes. The agent(s) may activate the cells of the cell population, for example. In some embodiments, a cell population, including an unmodified cell population and/or cell population activated by an agent, and/or a derivate of a cell population is administered to an individual with an agent. The administration may induce tissue differentiation for the reversing of tissue involution. The administration may induce tissue recruitment and differentiation of stems cells into epithelia cells of the tissue and/or fibroblasts of the tissue, such as splenic reticular fibroblasts. The administration may induce expansion of epithelia cells and/or fibroblasts present in a tissue.

In some embodiments, an agent comprises an adjuvant. In some embodiments, the adjuvant is administered to an individual to activate the immune system of the individual. The adjuvant may be used in combination with a cell population to activate the immune system. The adjuvants may be chemical, viral, bacterial product-based, or a combination thereof.

In certain embodiments, fibroblasts are cultured in vitro for preserving viability and proliferative ability of fibroblasts. The disclosure provides for the modification of known culture techniques to decrease recognition of fibroblasts by the recipient immune system. In one embodiment, fibroblasts are cultured in conditions that lack xenogeneic components, such as xenogeneic-free medium. In some cases, for example, the media is free of fetal calf serum. In specific embodiments, the disclosure encompasses the substitution of fetal calf serum with one or more other agents, such as those that facilitate reduction of immunogenicity of fibroblasts, for example, human platelet rich plasma, platelet lysate, umbilical cord blood serum, autologous serum, and/or one or more defined cytokines, such as one or a combination of fibroblast growth factor, epidermal growth factor, leukemia inhibitory factor, insulin like growth factor, angiopoietin, and vascular endothelial growth factor.

### A. Nucleic Acids

In certain embodiments, nucleic acid sequences can exist in a variety of instances such as: isolated segments and recombinant vectors of incorporated sequences or recombinant polynucleotides, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope are also provided. Nucleic acids encoding proteins, including fusion proteins are also provided. The nucleic acids can be single-stranded or double-stranded and can comprise RNA and/or DNA nucleotides and artificial variants thereof (e.g., peptide nucleic acids).

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### B. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5×sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### C. Mutation

Changes can be introduced by mutation into a nucleic acid including an endogenous or exogenous nucleic acid relative to any cell herein, thereby leading to changes in the amino acid sequence of a polypeptide (e.g., an antibody or antibody derivative) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### D. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of antibodies. See, eg., Gaily Kivi et al., BMC Biotechnol. 16:2 (2016). In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or part of one or more of the CDRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### E. Inhibitory Oligonucleotides

In some aspects, the disclosure relates to inhibitory oligonucleotides that inhibit the expression of a gene of interest. The inhibitory oligonucleotide may inhibit the RNA and/or protein expression of the gene of interest. Examples of an inhibitory oligonucleotides include but are not limited to siRNA (small interfering RNA), short hairpin RNA (shRNA), double-stranded RNA, an antisense oligonucleotide, a ribozyme, and an oligonucleotide encoding any thereof. An inhibitory oligonucleotide may inhibit the transcription of a gene or prevent the translation of a gene transcript in a cell. An inhibitory oligonucleotide acid may be from 16 to 1000 nucleotides long, and in certain embodiments from 18 to 100 nucleotides long. The oligonucleotide may have at least or may have at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, or 90 (or any range derivable therein) nucleotides. The oligonucleotide may be DNA, RNA, or a cDNA that encodes an inhibitory RNA.

Inhibitory oligonucleotides are well known in the art. For example, siRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

Particularly, an inhibitory oligonucleotide may be capable of decreasing the expression of the gene of interest by at least 10%, 20%, 30%, or 40%, more particularly by at least 50%, 60%, or 70%, and most particularly by at least 75%, 80%, 90%, 95%, 99%, or 100% more or any range or value in between the foregoing.

In further embodiments, there are synthetic oligonucleotides that inhibit a protein of interest. An inhibitor may be between 17 to 25 nucleotides in length and comprises a 5′ to 3′ sequence that is at least 90% complementary to the 5′ to 3′ sequence of a mature mRNA. In certain embodiments, an inhibitor molecule is 17, 18, 19, 20, 21, 22, 23, 24, or 25 nucleotides in length, or any range derivable therein. Moreover, an inhibitor molecule has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature mRNA, particularly a mature, naturally occurring mRNA. One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the inhibitory oligonucleotide is an analog and may include modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

The present disclosure concerns modified oligonucleotides, i.e., oligonucleotide analogs or oligonucleosides, and methods for effecting the modifications. These modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages are intended to replace naturally-occurring phosphodiester-5′-methylene linkages with four atom linking groups to confer nuclease resistance and enhanced cellular uptake to the resulting compound.

Modifications may be achieved using solid supports, which may be manually manipulated or used in conjunction with a DNA synthesizer using methodology commonly known to those skilled in DNA synthesizer art. Generally, the procedure involves functionalizing the sugar moieties of two nucleosides which will be adjacent to one another in the selected sequence. In a 5′ to 3′ sense, an “upstream” synthon such as structure H is modified at its terminal 3′ site, while a “downstream” synthon such as structure H1 is modified at its terminal 5′ site.

Oligonucleosides linked by hydrazines, hydroxylarnines, and other linking groups can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated DNA synthesis techniques. One of the most popular processes is the phosphoramidite technique. Oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass, TentaGel Support—an aminopolyethyleneglycol derivatized support or Poros—a copolymer of polystyrene/divinylbenzene. Attachment and cleavage of nucleotides and oligonucleotides can be effected via standard procedures. As used herein, the term solid support further includes any linkers (e.g., long chain alkyl amines and succinyl residues) used to bind a growing oligonucleoside to a stationary phase such as CPG. In some embodiments, the oligonucleotide may be further defined as having one or more locked nucleotides, ethylene bridged nucleotides, peptide nucleic acids, or a 5′(E)-vinyl-phosphonate (VP) modification. In some embodiments, the oligonucleotides has one or more phosphorothioated DNA or RNA bases.

### IV. Cellular Therapies

Certain embodiments herein encompass substantially homogeneous, homogeneous, or heterogeneous cell populations useful for indications disclosed herein. The cell population may comprise any cell disclosed herein, including modified and/or unmodified fibroblasts, modified and/or unmodified immune cells, or any modified and/or unmodified cell capable of regenerating a tissue, such as a spleen. Certain embodiments herein encompass methods for administering a cell population to an individual as a cellular therapy.

In some embodiments, fibroblasts are manipulated or stimulated to produce one or more factors and/or agents. In some embodiments, fibroblasts are manipulated or stimulated to produce leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-7, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and/or TGFβ-3. Factors from manipulated or stimulated fibroblasts may be present in conditioned media and collected for therapeutic use.

### A. Cell Culture

Certain embodiments concern culturing any suitable cells for incorporation into compositions and/or use in methods described herein. In some embodiments, cells are grown and maintained at an appropriate temperature, typically a temperature of 37° C. and under an atmosphere typically containing oxygen and CO₂. Culture conditions may vary widely for each cell type though, and variation of conditions for a particular cell type can result in different phenotypes being expressed. The most commonly varied factor in culture systems is the growth medium. Growth media can vary in concentration of nutrients, growth factors, and the presence of other components. The growth factors used to supplement media are often derived from animal blood, such as calf serum.

In some embodiments, cells may be cultured for at least between about 10 days and about 40 days, for at least between about 15 days and about 35 days, for at least between about 15 days and 21 days, such as for at least about 15, 16, 17, 18, 19 or 21 days. In some embodiments, the cells of the disclosure may be cultured for no longer than 60 days, or no longer than 50 days, or no longer than 45 days. The cells may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days. The cells may be cultured in the presence of a liquid culture medium. Typically, the medium may comprise a basal medium formulation as known in the art. Many basal media formulations can be used to culture cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the cells cultured. In some embodiments, a culture medium formulation may be explants medium (CEM) which is composed of IMDM supplemented with 10% fetal bovine serum (FBS), 100 U/ml penicillin G, 100 μg/ml streptomycin and 2 mmol/L L-glutamine. Other embodiments may employ further basal media formulations, such as chosen from the ones above.

Any medium capable of supporting cells in vitro may be used to culture the cells. Media formulations that can support the growth of cells include, but are not limited to, Dulbecco's Modified Eagle's Medium (DMEM), alpha modified Minimal Essential Medium (αMEM), and Roswell Park Memorial Institute Media 1640 (RPMI Media 1640) and the like. Typically, up to 20% fetal bovine serum (FBS) or 1-20% horse serum is added to the above medium in order to support the growth of cells. A defined medium, however, also can be used if the growth factors, cytokines, and hormones necessary for culturing cells are provided at appropriate concentrations in the medium. Media useful in the methods of the disclosure may comprise one or more compounds of interest, including, but not limited to, antibiotics, mitogenic compounds, or differentiation compounds useful for the culturing of cells. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing cells. Rather, any method of isolating and culturing cells should be construed to be included in the present disclosure.

For use in the cell culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In particular embodiments, cells are cultured in a cell culture system comprising a cell culture medium, preferably in a culture vessel, in particular a cell culture medium supplemented with a substance suitable and determined for protecting the cells from in vitro aging and/or inducing in an unspecific or specific reprogramming.

### B. Cell Generation

Certain methods of the disclosure concern culturing the cells obtained from human tissue samples. In particular embodiments of the present disclosure, cells are plated onto a substrate that allows for adherence of cells thereto. This may be carried out, for example, by plating the cells in a culture plate that displays one or more substrate surfaces compatible with cell adhesion. When the one or more substrate surfaces contact the suspension of cells (e.g., suspension in a medium) introduced into the culture system, cell adhesion between the cells and the substrate surfaces may ensue. Accordingly, in certain embodiments cells are introduced into a culture system that features at least one substrate surface that is generally compatible with adherence of cells thereto, such that the plated cells can contact the said substrate surface, such embodiments encompass plating onto a substrate, which allows adherence of cells thereto.

Cells of the present disclosure may be identified and characterized by their expression of specific marker proteins, such as cell-surface markers. Detection and isolation of these cells can be achieved, for example, through flow cytometry, ELISA, and/or magnetic beads. Single cell RNA-Seq and/or reverse-transcription polymerase chain reaction (RT-PCR) may be used to quantify cell-specific genes and/or to monitor changes in gene expression in response to differentiation. In certain embodiments, the marker proteins used to identify and characterize the cells are part of the list of cluster of differentiation (CD) markers that have been previously identified in publications for a variety of cell types and subtypes including immune systems cells. These can selected from the list consisting of c-Kit (CD117), Nanog, Sox2, Hey1, SMA, Vimentin, Cyclin D2, Snail, E-cadherin, Nkx2.5, GATA4, CD105, CD90, CD29, CD73, Wt1, CD34, CD45, CD4, CD8, CD3, TCR, CD2, CD28, CD5, CD7, CD40L, CD19, CD20, ldb, lga, sig, CD40, MHC II, CD22, CR1/CD35, CR2/CD21, B7/CD80, CD5, FcgR II/CD32, and a combination thereof.

### C. Polypeptide Expression

In some aspects, there are nucleic acid molecule encoding polypeptides or peptides of the disclosure, and any cells encompassed herein may be modified to produce them. These may be generated by methods known in the art, e.g., isolated from B cells of mice that have been immunized and isolated, phage display, expressed in any suitable recombinant expression system or by recombinant method. The nucleic acid molecules may be used to express large quantities of polypeptides.

### D. Vectors

In some aspects, contemplated are expression vectors comprising a nucleic acid molecule encoding a polypeptide of the desired sequence or a portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides may be inserted into expression vectors such that the gene area is operatively linked to transcriptional and translational control sequences. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### E. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### F. Methods of Gene Transfer

Suitable methods for nucleic acid delivery to effect expression of compositions are anticipated to include virtually any method by which a nucleic acid (e.g., DNA, including viral and nonviral vectors; CRISPR-Cas9) can be introduced into any cell, any tissue or any organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

In some embodiments, any cells encompassed herein may be modified to have knockdown or knockout of one or more endogenous genes. Those of skill in the art are familiar with standard procedures to do so, including RNA interference of any kind and CRISPR techniques, for example.

### G. CRISPR-Mediated Gene Activation

A variety of genes including Tumor necrosis factor receptor (TNFR) family, activating receptor for NFκB (RANK), CD40, lymphocyte p receptor (LTOR), Fibroblast growth factor (FGF) Foxn1, and Wnt in involved in the maturation and development of thymic epithelial cells (TECs)(Sun, Luo et al. 2013, Sun, Sun et al. 2015). The maturation of TECs have also been implicated in their interaction and dependence on other cells such as thymocytes, fibroblasts, and mesenchymal stem cells in that they can provide the necessary factors for epigenetic activation of genes necessary for maturation. In this embodiment, fibroblasts, and/or cells from the spleen can be epigenetically activated through CRISPR mediated methylation of the genes and promoter regions for one or a combination of the genes responsible for TEC activation destined for both the cortex and medulla of the thymus (Vojta, Dobrinid et al. 2016, Tyurin-Kuzmin, Karagyaur et al. 2018, Kang, Park et al. 2019, Nakamura, Gao et al. 2021, Singina, Sergiev et al. 2021). The same technique can be used for acetylation of the genes and promotor regions to reactivate the expression the genes necessary for the maturation of TECs.

### H. Host Cells

In another aspect, contemplated are the use of any host cells into which a recombinant expression vector has been introduced. An expression construct encoding a protein of interest can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the expression construct can be placed under control of a promoter that is linked to cell activation, such as one that is controlled by NFAT-1 or NF-1B. C One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### I. Formulations and Culture of the Cells

In particular embodiments, any cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, aMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain embodiments, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific embodiments, one or more of these may be explicitly excluded.

In some embodiments, the medium further comprises one or more vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27® supplement, GS21™ supplement, or combinations thereof. In some embodiments, the medium comprises or further comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21™ supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific embodiments, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific embodiments, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific embodiments, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

### V. Administration of Compositions

In one embodiment of the disclosure, effective amounts of fibroblasts as prepared in methods encompassed by the disclosure are administered to an individual for a therapy or prevention of one or more medical conditions. In specific embodiments, the fibroblasts are administered to improve endothelial responsiveness, increased immune cell activity, cell differentiation, and reversal of spleen atrophy.

In some embodiments, encapsulated RNA, microRNA, siRNA, or other RNAi compositions are administered with a cell population. The administration may locally activate tissue regeneration or reverse tissue involution.

Embodiments of the disclosure provide methods for co-administration of universal donor fibroblasts with one or more agents that stimulate immune cell activity in the spleen [9, 10]. In a specific embodiment of the disclosure, methods are provided for co-administration of universal donor fibroblasts with one or combination of growth factors, chemokines, and/or cytokines. In one embodiment of the disclosure, universal donor fibroblasts derived from fibroblasts that have been treated under conditions to reduce immunogenicity are utilized to stimulate vascular endothelial growth factor (VEGF) production by the introduced modified/unmodified fibroblasts or from endogenous cells under the regulatory control of nerve growth factor (NGF) of the individual thereby leading to tissue regeneration and revascularization of the spleen as it has been demonstrated with other organs such as thymus [11-13].

The therapy provided herein may comprise administration of a therapeutic compositions (e.g., fibroblasts, immune cells, derivatives of cell, exosomes from fibroblasts, agents, etc.) alone or in combination. Therapies may be administered in any suitable manner known in the art. For example, a first and second treatment may be administered sequentially (at different times) or concurrently (at the same time). In some embodiments, the first and second treatments are administered in a separate composition. In some embodiments, the first and second treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the compositions may be employed.

The therapeutic compositions (e.g., cells, cell products, including fibroblasts or from fibroblasts) of the disclosure may be administered by the same route of administration or by different routes of administration. In some embodiments, the therapy is administered intravenously, intramuscularly, subcutaneously, topically, orally, transdermally, intraperitoneally, intraorbitally, by implantation, by inhalation, intrathecally, intraventricularly, or intranasally. In some embodiments, the composition is administered directly to the spleen. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In some embodiments, between about 10⁵ and about 10¹³ cells per 100 kg are administered to a human per infusion. In some embodiments, between about 1.5×10⁶ and about 1.5×10¹² cells are infused per 100 kg. In some embodiments, between about 1×10⁹ and about 5×10¹¹ cells are infused per 100 kg. In some embodiments, between about 4×10⁹ and about 2×10¹¹ cells are infused per 100 kg. In some embodiments, between about 5×10⁸ cells and about 1×10¹¹ cells are infused per 100 kg. In some embodiments, a single administration of cells is provided. In some embodiments, multiple administrations are provided. In some embodiments, multiple administrations are provided over the course of 3-7 consecutive days. In some embodiments, 3-7 administrations are provided over the course of 3-7 consecutive days. In some embodiments, 5 administrations are provided over the course of 5 consecutive days. In some embodiments, a single administration of between about 105 and about 10¹³ cells per 100 kg is provided. In some embodiments, a single administration of between about 1.5×10⁸ and about 1.5×10¹² cells per 100 kg is provided. In some embodiments, a single administration of between about 1×10⁹ and about 5×10¹¹ cells per 100 kg is provided. In some embodiments, a single administration of about 5×10¹⁰ cells per 100 kg is provided. In some embodiments, a single administration of 1×10¹⁰ cells per 100 kg is provided. In some embodiments, multiple administrations of between about 10⁵ and about 10¹³ cells per 100 kg are provided. In some embodiments, multiple administrations of between about 1.5×10⁸ and about 1.5×10¹² cells per 100 kg are provided. In some embodiments, multiple administrations of between about 1×10⁹ and about 5×10¹¹ cells per 100 kg are provided over the course of 3-7 consecutive days. In some embodiments, multiple administrations of about 4×10⁹ cells per 100 kg are provided over the course of 3-7 consecutive days. In some embodiments, multiple administrations of about 2×10¹¹ cells per 100 kg are provided over the course of 3-7 consecutive days. In some embodiments, 5 administrations of about 3.5×10⁹ cells are provided over the course of 5 consecutive days. In some embodiments, 5 administrations of about 4×10⁹ cells are provided over the course of 5 consecutive days. In some embodiments, 5 administrations of about 1.3×10¹¹ cells are provided over the course of 5 consecutive days. In some embodiments, 5 administrations of about 2×10¹¹ cells are provided over the course of 5 consecutive days.

### VI. Kits of the Disclosure

Any of the cellular and/or non-cellular compositions described herein or similar thereto may be comprised in a kit. In a non-limiting example, one or more reagents for use in methods for preparing fibroblasts, fibroblast-derived products, or derivatives thereof (e.g., exosomes derived from fibroblasts) may be comprised in a kit. Such reagents may include cells, vectors, one or more growth factors, vector(s) one or more costimulatory factors, media, enzymes, buffers, nucleotides, salts, primers, compounds, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

Although the present disclosure and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

The following references, to the extent that they provide exemplary procedural or other details supplementary to those set forth herein, are specifically incorporated herein by reference.
1. Clark, R. A. and T. S. Kupper, IL-15 and dermalfibroblasts induce proliferation of natural regulatory T cells isolated from human skin. Blood, 2007. 109(1): p. 194-202.
2. Quandelacy, T. M., et al., Age- and sex-related risk factors for influenza-associated mortality in the United States between 1997-2007. Am J Epidemiol, 2014. 179(2): p. 156-67.
3. Tan, J. K. and T. Watanabe, Murine spleen tissue regeneration from neonatal spleen capsule requires lymphotoxin priming of stromal cells. J Immunol, 2014. 193(3): p. 1194-203.
4. Tan, J. K. H. and T. Watanabe, Determinants of postnatal spleen tissue regeneration and organogenesis. NPJ Regen Med, 2018. 3: p. 1.
5. Turner, V. M. and N. A. Mabbott, Influence of ageing on the microarchitecture of the spleen and lymph nodes. Biogerontology, 2017. 18(5): p. 723-738.
6. Sun, L., et al., FSP1(+) fibroblast subpopulation is essential for the maintenance and regeneration of medullary thymic epithelial cells. Sci Rep, 2015. 5: p. 14871.
7. Riera, M., S. Buczacki, and Z. A. Khan, Splenic regeneration following splenectomy and impact on sepsis: a clinical review. J R Soc Med, 2009. 102(4): p. 139-42.
8. Han, D., et al., Targeting Brain-spleen Crosstalk After Stroke: New Insights Into Stroke Pathology and Treatment. Curr Neuropharmacol, 2021. 19(9): p. 1590-1605.
9. Bronte, V. and M. J. Pittet, The spleen in local and systemic regulation of immunity. Immunity, 2013. 39(5): p. 806-18.
10. Lewis, S. M., A. Williams, and S. C. Eisenbarth, Structure and function of the immune system in the spleen. Sci Immunol, 2019. 4(33).
11. Park, H. J., et al., Up-regulation of VEGF expression by NGF that enhances reparative angiogenesis during thymic regeneration in adult rat. Biochim Biophys Acta, 2007. 1773(9): p. 1462-72.
12. Zanardo T É, C., et al., Decellularized Splenic Matrix as a Scaffold for Spleen Bioengineering. Front Bioeng Biotechnol, 2020. 8: p. 573461.
13. Smith, J. N., et al., 15-PGDH inhibition activates the splenic niche to promote hematopoietic regeneration. JCI Insight, 2021. 6(6).
14. Lim, H. K. and H. C. O'Neill, Identification of Stromal Cells in Spleen Which Support Myelopoiesis. Front Cell Dev Biol, 2019. 7: p. 1.
15. Wang, Z., et al., The spleen may be an important target of stem cell therapy for stroke. J Neuroinflammation, 2019. 16(1): p. 20.
16. Faustman, D. L., Regenerative medicine: Stem cell research turns to the spleen. Discov Med, 2005. 5(29): p. 447-9.
17. Faustman, D. L. and M. Davis, Stem cells in the spleen: therapeutic potential for Sjogren's syndrome, type I diabetes, and other disorders. Int J Biochem Cell Biol, 2010. 42(10): p. 1576-9.

What is claimed is:

1. A method of regenerating a spleen or tissue of a spleen in an individual, comprising the step of administering an effective amount of a population of cells to the individual, wherein the population of cells comprises unmodified or modified fibroblasts, unmodified or modified immune cells, activated or non-activated fibroblasts, activated or non-activated immune cells, fibroblast derivatives, immune cell derivatives, or a combination thereof, thereby resulting in regeneration of spleen tissue in the individual.

2. The method of claim 1, wherein the fibroblast derivative comprises extracellular vesicles, exosomes, microvesicles, apoptotic bodies, or any other fragments or biologic components of fibroblast cells.

3. The method of claim 1 or 2, wherein prior to and/or during the administering, the population of cells is activated by exposing the cells to one or more agents capable of activating the cells.

4. The method of claim 3, wherein the agent(s) comprise one or more nucleic acids, cytokines, chemokines, transcription factors, epigenetic factors, growth factors, hormones, or a combination thereof.

5. The method of any one of claims 1-4, wherein the population of cells is activated in vitro, in vivo, or ex vivo.

6. The method of any one of claims 1-5, wherein the population of cells are autologous, allogeneic, xenogeneic, syngeneic, or a combination thereof, with respect to the individual.

7. The method of any one of claim 1-6, further comprising administering one or more adjuvants to the individual.

8. The method of claim 7, wherein the adjuvant comprises a chemical adjuvant, a viral adjuvant, a bacteria adjuvant, or a combination thereof.

9. The method of claim 7 or 8, wherein the adjuvant comprises an encapsulated RNA, a microRNA, an siRNA, or a combination thereof.

10. The method of any one of claims 1-9, wherein the regenerating comprises organogenesis and/or T cell development and/or B cell development.

11. The method of any one of claims 1-10, wherein the regenerating comprises reversing of splenic atrophy.

12. The method of any one of claims 1-11, wherein the regenerating comprises reinvigorating or stimulating of immune cell production in the spleen.

13. The method of any one of claims 1-12, wherein the regenerating comprises spleen differentiation.

14. The method of any one of claims 1-13, wherein the regenerating comprises spleen recruitment, differentiation of stems cells into splenic epithelia cells and/or splenic fibroblasts, and/or expansion of splenic epithelia cells and/or splenic fibroblasts.

15. The method of claim 14, wherein the splenic fibroblasts comprise splenic reticular fibroblasts.

16. The method of any one of claims 1-15, wherein the activated population of cells is administered systemically to the individual.

17. The method of any one of claims 1-15, wherein the activated population of cells is administered locally to the spleen of the individual.

18. An in vitro method of producing activated or non-activated fibroblasts, derivatives of activated fibroblasts, activated or non-activated immune cells, derivatives of activated or non-activated immune cells, or a combination thereof, comprising the step of exposing fibroblasts and/or immune cells to one or more conditions that induce activation, wherein the activated fibroblasts, derivatives of activated fibroblasts, activated immune cells, and/or derivatives of activated immune cells comprise activity for regenerating spleen tissue.

19. The method of claim 18, wherein the activated or non-activated fibroblast cells and/or activated or non-activated immune cells are autologous to the spleen.

20. The method of claim 18 wherein the activated or non-activated fibroblast cells and/or activated or non-activated immune cells are allogeneic to the spleen.

21. The method of claim 18, wherein the activated fibroblast cells and/or activated immune cells are xenogeneic to the spleen.

22. The method of claim 18, wherein the activated fibroblast cells and/or activated immune cells are syngeneic to the spleen.

23. The method of any one of claims 18-22, wherein the regenerating comprises stimulating, improving, or increasing production and/or activity of immune cells in a spleen.

24. The method of any one of claims 18-21, wherein the one or more conditions comprise one or more nucleic acids, cytokines, chemokines, transcription factors, epigenetic factors, growth factors, hormones, or a combination thereof.

25. A method of stimulating, improving, or increasing production and/or activity of cells in a spleen in an individual, comprising the step of providing to the individual an effective amount of activated fibroblasts, derivatives of activated fibroblasts, activated immune cells, derivatives of activated immune cells, or a combination thereof.
